(19) United States
(12) Patent Application Publication
(10) Pub. No.: US 2024/0115743 A1
(43) Pub. Date: Apr. 11, 2024

(54) INSTANTLY GELLING COMPOSITIONS FOR ARTIFICIAL SALIVA AND ULTRASOUND GELS

(71) Applicant: KONINKLIJKE PHILIPS N.V., Eindhoven (NL)

(72) Inventors: Jingliang MAO, Shoreline, WA (US); Gregory Russ GODDARD, Kenmore, WA (US)

(21) Appl. No.: 18/267,136

(22) PCT Filed: Dec. 14, 2021
(86) PCT No.: PCT/EP2021/085587

§ 371 Date: Jun. 14, 2023

Related U.S. Application Data
(60) Provisional application No. 63/126,342, filed on Dec. 16, 2020.

(51) Int. Cl. A61K 49/22

(52) U.S. Cl. CPC A61K 49/226

(57) ABSTRACT

A method (100) for generating an instantly-formed artificial saliva gel or an ultrasound gel, comprising: (i) providing (110) a first composition comprising a pH-sensitive polymer, wherein the first composition is maintained at an acidic pH; (ii) providing (110) a second composition comprising a pH agent configured to maintain the second composition at an alkaline pH; and (iii) combining (120) the first composition and the second composition to generate a single instantly-formed gel, wherein the pH of the single instantly-formed gel is at a level required to increase viscosity of the pH-sensitive polymer to a level suitable for an artificial saliva gel or an ultrasound gel; wherein either the first composition or second composition comprises a solvent, and wherein the acidic pH of the first composition and the alkaline pH of the second composition are configured such that the pH of the single instantly-formed gel is between approximately 6.5 and 7.5.

### FIELD OF THE DISCLOSURE

The present disclosure is directed generally to methods and systems for generating an instantly-formed artificial saliva gel or an ultrasound gel just prior to use.

### BACKGROUND

Dry mouth, or xerostomia, is a chronic condition in salivary glands don't make enough saliva to keep the mouth wet. It is a symptom associated with a wide variety of causes and conditions such as certain medications, aging, dehydration, chemotherapy, radiation therapy, or diseases such as Sjögren's syndrome, rheumatoid arthritis, scleroderma, diabetes mellitus, and hypertension. Dry mouth may also be caused by psychogenic reasons or by a condition that directly affects the salivary glands.

Dry mouth can range from being merely a nuisance to something that has a big impact on teeth and gum health, general health, as well as appetite and enjoyment of food. For example, dry mouth could be seen as a chronic disorder which may lead to numerous health, nutritional, and social hazards. Sores often develop in dry areas inside the mouth of those afflicted. Speech may become strained and painful, which may lead sufferers to withdraw from social contact. Even breathing could become a difficult task. Dry mouth is also responsible for secondary effects, including an increased incidence of dental caries and demineralization of teeth, and increased risk of secondary infections such as candidiasis. Dry mouth leads to the inability to enjoy a standard of living taken for granted by most. Although dry mouth occurs in people of all ages, it is an especially common complaint in elderly people, and affect approximately 20% of the elderly. It is estimated that millions of people In USA have some kind of dry mouth with different severity.

For the treatment of dry mouth itself, artificial saliva or saliva substitutes can be used to lubricate the mouth, moisten the mouth, and replenish internal surface of mouth with moisturizer. These substitutes are available in a variety of formulations including solutions, sprays, gels and lozenges.

In ultrasound imaging and/or therapy, tiny hairs and dry skin hold many little pockets of air, and sound waves do not travel through the air very well. An ultrasound gel is a necessity as it serves as a connector and establishes a bond between the skin and the ultrasound probe by eliminating the air, helping the sound waves travel directly to the tissues beneath the skin of the patient and enabling a clear image of the organ of interest. For better scan results, an ultrasound gel is used. In composition, the gel is specially formulated to act as a coupling agent and reduce static while using ultrasound equipment. A key requirement of this gel is to have acoustic impedance similar to that of soft tissue. The gel need to be maintained its gel property when heated by the body temperature. In internal ultrasound imaging and/or therapy, it is common that use of a gel with ultrasonic dental therapy or imaging devices requires the patient to apply the gel intra-orally, which can lead to ingestion of small quantities of gel. Thus food grade internal ultrasound gel is desired even it is optional.

While current ultrasound gels and artificial saliva gels may function for their intended purpose, there is a continuing desire to improve them further for better performance from a cost-effective and convenience perspective.

### SUMMARY OF THE DISCLOSURE

Accordingly, there is a continued need in the art for ultrasound gels and artificial saliva gels that are more convenient and more cost-effective.

The present disclosure is directed to inventive instantly-formed ultrasound gels and artificial saliva gels. Various embodiments and implementations herein are directed to a method or kit configured to generate an instantly-formed artificial saliva gel or an ultrasound gel just prior to use of the gel. A first composition is provided, the first composition comprising a pH-sensitive polymer, and where the first composition is maintained at an acidic pH. A second composition is provided, the second composition comprising a pH agent configured to maintain the second composition at an alkaline pH. The first composition and the second composition are combined to generate a single mixture, which is an instantly-formed gel via mixing the two compositions together through a mixing tip or mixing chamber. The pH of the single mixture is above a threshold required to increase viscosity of the pH-sensitive polymer and the single mixture to a level suitable for an artificial saliva gel or an ultrasound gel. Either the first composition or second composition comprises a solvent, and wherein the acidic pH of the first composition and the alkaline pH of the second composition are configured such that the pH of the single mixture is between approximately 6.5 and 7.5. The composition may thus be prepared in situ by combining, e.g. mixing, the respective components. The adjustment of the composition to pH 6.5-7.5 may result in the composition becoming a gel-like composition, which can be regarded simply as a “gel composition.” In this case, the term “gel” merely refers to the pH-induced swelling of the polymer, which will be described in more detail herein below. The gel composition may be formed instantly or instantaneously when the composition has the pH 6.5-7.5. The terms “instantly” or “instantaneously” in this context may mean that the kinematic viscosity of the composition increases in response to adjustment to the desired pH in a time period which is, for example, less than 30 seconds, such as less than 10 seconds, and preferably less than 3 seconds from the pH adjustment at room temperature (˜20° C.).

Generally, according to an embodiment, a method for generating an instantly-formed artificial saliva gel or an ultrasound gel just prior to use of the gel is provided. The method includes: (i) providing a first composition comprising a pH-sensitive polymer, such as HASE (Hydrophobically-modified Alkali Soluble Emulsion) polymer, wherein the first composition is maintained at an acidic pH; (ii) providing a second composition comprising a pH agent configured to maintain the second composition at an alkaline pH; and (ii) combining the first composition and the second composition to generate a single mixture (which is an instantly-formed gel), wherein the pH of the single mixture is at a level required to increase viscosity of the pH-sensitive polymer to a level suitable for an artificial saliva gel or an ultrasound gel; wherein either the first composition or second composition comprises a solvent, and wherein the acidic pH of the first composition and the alkaline pH of the second composition are configured such that the pH of the single mixture/gel is between approximately 6.5 and 7.5.

According to an embodiment, the method further includes providing a third composition comprising a flavor agent and/or a surfactant, where the combining step further comprises combining the third composition with the first composition and the second composition.

According to an embodiment, the method further includes providing a third composition, wherein the third composition rather than the first composition or the second composition comprises the solvent, where the combining step further comprises combining the third composition with the first composition and the second composition.

According to an embodiment, the pH agent is a hydroxide, carbonate, bicarbonate, silicate, phosphate, and/or variations thereof.

According to an embodiment, the first composition or second composition comprises a flavor agent, a surfactant, a sweetening agent, and/or a fragrance. According to an embodiment, the agent is xylitol, menthol, eugenol, and/or an essential oil. According to an embodiment, the surfactant is sodium lauryl sulfate (SLS).

According to an embodiment, the solvent is propylene glycol and/or glycerin.

According to an embodiment, the first composition and the second composition do not comprise a preservative.

According to a second aspect is a kit configured to generate an instantly-formed artificial saliva gel or an ultrasound gel just prior to use of the gel. The kit includes: (i) a first composition comprising a pH-sensitive polymer, wherein the first composition is maintained at an acidic pH; and (ii) a second composition comprising a pH agent configured to maintain the second composition at an alkaline pH; where combining the first composition and the second composition generates a single mixture which is an instantly-formed gel, wherein the pH of the single mixture is at a level required to increase viscosity of the pH-sensitive polymer to a level suitable for an artificial saliva gel or an ultrasound gel; and where the first composition or second composition comprises a solvent, and wherein the acidic pH of the first composition and the alkaline pH of the second composition are configured such that the pH of the single mixture or instantly-formed gel is between approximately 6.5 and 7.5.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for generating an instantly-formed artificial saliva gel or an ultrasound gel, in accordance with an embodiment.

FIG. 2 is a schematic representation of a dispensing system, in accordance with an embodiment.

### DETAILED DESCRIPTION OF EMBODIMENTS

The present disclosure describes various embodiments of a method or kit configured to generate an instantly-formed artificial saliva gel or an ultrasound gel just prior to use of the gel. More generally, Applicant has recognized and appreciated that it would be beneficial to provide a system for convenient and cost-effective storage, shipping, and use of an artificial saliva gel or an ultrasound gel. A first composition is provided, the first composition comprising a pH-sensitive polymer, and where the first composition is maintained at an acidic pH. A second composition is provided, the second composition comprising a pH agent configured to maintain the second composition at an alkaline pH. The first composition and the second composition are combined via a mixing tip/chamber with turbulent or tortuous path to ensure complete mixing to generate a single mixture, which is an instantly-formed gel. The pH of the single mixture is above a threshold required to increase viscosity of the pH-sensitive polymer to a level suitable for forming an instantly-formed an artificial saliva gel or an ultrasound gel. Either the first composition or second composition comprises a solvent, and wherein the acidic pH of the first composition and the alkaline pH of the second composition are configured such that the pH of the single mixture/gel is between approximately 6.5 and 7.5.

Accordingly, the methods and systems described or otherwise envisioned herein provided an instant in-situ gelling technology that produced as both an ultrasound gel and an artificial saliva gel. Compared to current methods or technology to produce pre-formed ultrasound gels and dry mouth gels separately, one aim of embodiments herein is to produce a gel that can be used for both ultrasound and dry mouth applications. Embodiments comprise an improved gel production process, simpler packaging, and superior performance. Embodiments focus on producing high quality oral gels while simplifying the gel production process. Embodiments are intended for dry mouth relief and oral internal ultrasound scanning/therapy in oral healthcare, but the instantly-gelling approach can be readily expanded to other personal care gel products, which covers household, industrial, institutional, health care and other areas.

The embodiments disclosed herein provide numerous advantages. For example, market-available preformed ultrasound gels may comprise air bubbles. Air bubbles of different sizes, especially small sizes of air bubbles, may be generated and trapped in the current dry mouth relief or oral internal ultrasound gels during production. Those air bubbles in the gels affect product aesthetic appearance and quality, especially those mini-air bubbles in the gel may affect getting clear image of the organ(s) of interest during internal ultrasound imaging. Another example includes the need for tuning the acoustic impedance in the form of sound speed or viscosity to achieve better ultrasonic imaging of different tissues with different probe styles (i.e., intercavity vs. curved vs. linear). Viscosity tuning is also relevant to achieving appropriate organoleptic experience for artificial saliva applications.

Additionally, the blending, filling, packaging and storage of pre-formed gels are cumbersome due to the viscous nature of the gels. It often requires controlled production conditions and measures, including vacuum treatment to remove trapped as many air bubbles as many as possible. It also requires special filling/packaging equipment to package those viscous gels into the final containers. Many pre-formed ultrasound or artificial saliva gels need special preservative(s) and/or storage conditions such as refrigeration to keep them fresh. All those ingredients, conditions, measures and special equipment inevitably lead to higher cost in gel production, packaging and storage. For pre-formed gels stored at room temperature or bulk gel in opened container, potential microorganisms' contamination will always exist due to approximately neutral pH gel environment. This may cause shelf life issue to finished gel products. Due to high viscous nature of gel and potential air-bubble problem, it has been always an issue to achieve an accurate dosing of the amount of gel needed. Most types of gel flavorings are focused on scent and taste. Many gel flavorants are ester type derivatives and are more or less hydrophobic. Since the overall property of ultrasound gel or artificial saliva gel tend to be hydrophilic, this creates a compatibility issue for flavor agents to be homogenous inside the gel. It often requires addition of surfactants and coupling agents to make flavor highly soluble in a hydrophilic environment. Thus it makes the choice of flavors in pre-formed gel less than instantly in-situ formed gel, in which the latter usually requires the flavor agent(s) able to stay with gel for less than a couple of hours.

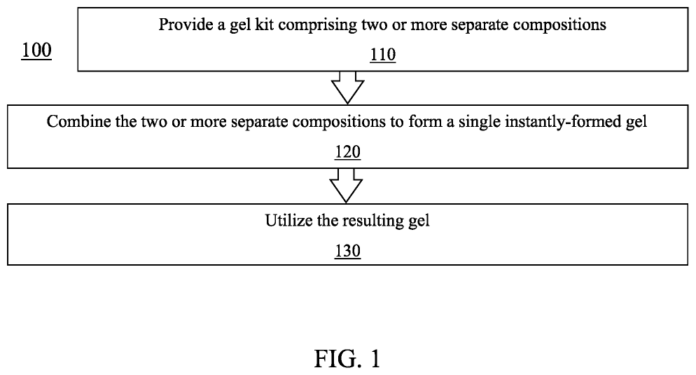

Referring to FIG. 1, in one embodiment, is a flowchart of a method 100 configured to generate an instantly-formed artificial saliva gel or an ultrasound gel just prior to use of the gel. The methods described in connection with the figures are provided as examples only, and shall be understood not to limit the scope of the disclosure.

At step 110, an artificial saliva gel kit or an ultrasound gel kit is provided. The artificial saliva gel kit or ultrasound gel kit can be any of the kits described or otherwise envisioned herein. According to an embodiment, the kit comprises multiple components.

According to an embodiment, the kit comprises a first composition with a pH-sensitive polymer, where the first composition is maintained at an acidic pH. The pH-sensitive polymer can be any polymer suitable for the gel kit. For example, the pH-sensitive polymer can be an alkali-swellable emulsion (ASE) polymer, a hydrophobically-modified alkali soluble emulsion (HSAE) polymer, a hydroxyethylcellulose (HEC) polymer, or any other suitable polymer.

According to an embodiment, when the pH of the final composition with pH sensitive thickening polymer is above 6.5, especially above 7.0, the final composition immediately thickens exponentially to become highly viscous, such as, for example, 50,000-100,000 mPa. When the pH of the final composition is below 7.0, especially when the pH is below 6.5 or lower, it remains low viscosity, such as <100 mPa, and preferably <50 mPa.

The pH-sensitive polymer can be any polymer suitable for the gel kit. According to an embodiment, the pH-sensitive polymer is an alkali-swellable emulsion (ASE) polymer. ASE polymers have groups, such as sulfonic acids (—SO₃H), phosphonic acids, boronic acids, amino acids, or carboxyl groups, which are deprotonated at alkaline pH. This deprotonation causes the aqueous solubility of the polymer to increase, such that the degree of swelling of the polymer at the alkaline pH is far greater than the degree of swelling at an acidic pH, which is less than 7, or often less than 4, which is desirable for instantly in-situ formed gels in this invention. This greater degree of swelling at slightly alkaline pH causes a dramatic increase in the kinematic viscosity of the constituent, which includes the polymer, such that the constituent becomes gel-like.

One non-limiting example of an ASE polymer is Carbopol® 940 NF polymer. The pH of a 1% solids dispersion solution of Carbopol 940 NF is 2.5-3.0, and the viscosity is well below 1500 cps. However, under pH 7.5-10.0, the polymer results in 30,000-40,000 cps of viscosity at 0.5% solids dispersions at 20 rpm. A 1% solids dispersion of neutralized mucilage of Carbopol 940 NF polymer forms a stiff gel. According to an embodiment, the dosage of Carbopol 940 NF and other suitable Carbopol polymers is between 0.5-3% level in the final mixed gel, although other dosages are possible.

According to an embodiment, the pH-sensitive polymer is a hydrophobically-modified alkali soluble emulsion (HSAE) polymer. HSAE polymers consist of acid/acrylate copolymer backbone and ethoxylated hydrophobe. The pH-sensitive gelling mechanism of HASE polymers is not only created by the electrostatic repulsion among charges present on the polymer chain, but also due to networking of numerous pendant hydrophobic groups present in their molecules. As pH-sensitive rheology modifiers, HASE polymer swells first due to charge-charge repulsion when its acid groups are neutralized/deprotonated with inorganic bases or organic amines, and therefore thickens instantly in response to an increased pH. With these polymers swelling, the pendant hydrophobic groups are then freed to build associations with one another and with other hydrophobes available in the formulation, such as surfactants, particulates, emulsion droplets and dyes. This phenomenon creates a network structure that results in a significant viscosity increase, which further boosts the thickening/gelling effect and stabilizes/disperses any particulates which may be present in the formulation. At the same dosage level and similar molecular weight, HASE polymers thicken faster and more viscous than ASE polymers. HASE polymers have better salt compatibility than ASE polymers.

One non-limiting example of HASE polymers is Acusol® 820, which forms a viscous gel, such as >100,000 cps at 6 rpm, at pH 7.0-12.0 with 1.5% wt. solids dosage. Very little viscosity, such as <30 cps for 100% Acusol® 820, 30% solids content, is exhibited at pH 3.0. Another example is Aculyn® 28, which has low viscosity of 20 cps with pH 3.0 at 20% solids content. However, at 25° C., when the pH goes between 6.5 and 8.0, viscosity of 1% solids of Aculyn 28 is approximately 100,000 cps at 5 rpm. According to an embodiment, the dosage level of Acusol® 820, Aculyn 28, and/or other HASE polymers is between 1%-6% solids level in the final mixed gel, from a cost-effect perspective, although other dosages are possible. According to an embodiment, the pH-sensitive polymer is a hydroxyethylcellulose (HEC) polymer. Polymerization is accomplished hydration of hydroxyl groups by forming hydrogen bonds with water molecules and among themselves via water molecule as bridges. A slightly alkaline pH close to pH 7.0 dramatically enhances the wetting/dispersion speed of hydroxyl groups in HEC, thus the gelling speed. In addition, using glycerin as viscosity enhancer in the formula and the networking of numerous pendant hydroxyethyl groups present in HEC molecules boosts the viscosity further when HEC molecules begin to swell/jellify with hydration.

According to an embodiment, the kit comprises a second, separate composition. The second composition comprises a pH agent configured to maintain the second composition at an alkaline pH. According to an embodiment, in order to attain the desired pH in a final combined mixture, an alkaline pH modifying agent solution is used in the second composition. The pH modifying agent can be attained by the use of alkali earth or metal hydroxides, particularly potassium and sodium hydroxides, and/or other suitable compound. Carbonates, bicarbonates, and/or other compatible chemicals can also be used for formulation in the alkaline composition of the second composition, as can silicates, phosphates, and/or other buffers. Preferably, the alkaline pH modifying agent is a food-grade agent and is safe to humans when it is neutralized with the acidic solution of the first composition, to approximately pH 6.0-8.0.

At step 120 of the method, the first composition and the second composition are combined via a mixing tip/chamber with turbulent or tortuous path to ensure complete mixing to create a single mixture, which is an instantly-formed gel. The acidity of the first solution and the alkalinity of the second solution are neutralized, which results in gelation of the pH-sensitive polymer. According to an embodiment, the pH of the combined single mixture is to result in a pH is at a level required to increase viscosity of the pH-sensitive polymer to a level suitable for an artificial saliva gel or an ultrasound gel. For example, the pH of the combined single mixture (gel) may be approximately 6.5-8.0.

The first composition and the second composition can be combined in any manner that results in a single instantly-formed gel. The first composition may be added to the second composition, the second composition may be added to the first composition, or the two compositions may be combined in another container, body, or other element.

According to an embodiment, the mixing ratio of the first composition and the second composition is designed to arrive at a desired amount, pH, and/or viscosity. According to just one embodiment, the mixing ratio of the two compositions is in the range 1:1 to 10:1, or 1:0 to 1:10, with ratios around 1:1 to 4:1 or 1:1 to 1:4 being particularly preferred. It will be appreciated that the optimal mixing ratio will be dependent upon the nature and quantities of the components present. According to an embodiment, the desired operative consideration being that the final mixed composition should have a pH within pH 6.5-8, and preferably pH 6.5-7.5 for certain applications.

According to an embodiment, one or both of the first composition and the second composition comprises another agent, such as a flavoring agent, a fragrance agent, a surfactant, a solvent, a sweetening agent, or any other desirable agent.

According to an embodiment, one or both of the first composition and the second composition comprises a solvent. The solvent can be any non-toxic solvent providing one or more qualities desired in the single mixture. For example, the solvent may be propylene glycol and/or glycerin, although many other solvents are possible.

According to an embodiment, one or both of the first composition and the second composition comprises a surfactant. The surfactant can be any non-toxic surfactant providing one or more qualities desired in the single mixture. For example, the surfactant may be sodium lauryl sulfate (SLS) and/or an SLS-related surfactant, although many other surfactants are possible.

According to an embodiment, one or both of the first composition and the second composition comprises a flavoring agent. The flavoring agent can be any flavoring agent providing one or more qualities desired in the single mixture. For example, the flavoring agent is utilized to improve consumer perception and experience in treatment/application by either adding flavor to an otherwise flavorless product or to mask any undesirable flavors. Generally, for oral products, minty flavors are used to give a sense of cleanliness and freshness. Sweeteners are also used but not to the same extent as other consumer products as sweetness is perceived to be detrimental to dental health. For example, flavor agents such as menthol or peppermint oil is used for artificial saliva and/or ultrasound gels applications. Eugenol may also be used.

According to an embodiment, one or both of the first composition and the second composition comprises a sweetening agent. The sweetening agent can be any sweetening agent providing one or more qualities desired in the single mixture. For example, the sweetening agent is xylitol, although many other sweetening agents are possible. Xylitol is a non-fermentable sugar alcohol flavoring agent and has anti-microbial property.

According to an embodiment, when the flavoring agent, fragrance agent, surfactant, solvent, or other desirable agent is maintained in the first composition, the agent is low pH compatible but functions at the pH of the single mixture.

Any method of combining the first composition and the second composition to produce a single combined mixture is possible. According to one embodiment, the combination system can be a dual-barrel syringe, a dual-barrel bottle, or a dispensing device that can dispense two or more separate fluids to a common mixing tip/chamber. Such a dual-barrel syringe, dual-barrel bottle, or dispensing device can be formed with two compartments, such as detached containers/cartridges or integrated chambers in the device, that provide dual reservoirs for the parts of fluids to be combined at the mixing ratio as desired. The apparatus further can comprise pump or vacuum means to simultaneously withdraw fluid from each compartment, via separate draw pipes, and discharge the fluid to a common mixing tip or chamber and after that, to the application areas. This device enables the alkaline and the acidic parts of materials to be stored separately before use. When needed, the alkaline and the acidic components are mixed and gelled from a single unit at a joint mixing point or chamber before starting of applications.

For applications, the on-site instantly formed gels can also be transferred and dispensed onto targeted area(s) by syringe, extruder, spray bottle, pipette, tray, brush, strips, or other suitable devices. In addition, any tray or strip that can carry the final artificial saliva gel and/or internal ultrasound gels to the target works.

At step 130 of the method, the resulting gel is utilized as an artificial saliva gel, an ultrasound gel, or for another intended purpose.

Referring to TABLE 1 is one possible embodiment for a two-component kit or system, with a first composition and a second composition. According to an embodiment, the mix ratio of components in TABLE 1 is 1:1 (v/v) to create a final instantly-formed artificial saliva or internal ultrasound gel with ˜45,000 cps @pH 6.5-8.5, 10 rpm and acoustic impedance around 1.5 MRayl, although other variations are possible. The compositions are provided only as an example, and it will be understood that it does not limit the scope of the methods and kits described or otherwise envisioned herein.

TABLE 1
Possible components of a two-component kit or system.

| Ingredient | |
|---|---|
| | % w/w in the |
| | second composition |
| DI Water | 63.54 |
| Potassium Hydroxide | 0.46 |
| Propylene Glycol | 18.00 |
| Glycerin | 18.00 |
| | % w/w in the |
| | first composition |
| DI Water | 77.90 |
| Xylitol | 2.00 |
| ACULYN 28 | 10.00 |
| Glycerin | 10.00 |
| Nat. Peppermint Oil PE-5523 | 0.01 |
| Sodium Lauryl Sulfate (30%) | 0.09 |

According to an embodiment, the single instantly-formed gel is formed by combining more than two different compositions via a complete mixing through a mixing tip or chamber. For example, the method or kit may comprise combining three, four, five, or more compositions to form a single instantly-formed gel.

According to an embodiment, the single instantly-formed gel is formed by combining three different compositions via a complete mixing through a mixing tip or chamber. Other functional ingredients can be formulated into one part in the third composition. In addition, the ingredient concentration of each part may be higher than the two-part composition system. For a three-part composition, the dosing system can be a tri-barrel syringe, tri-chamber bottle, and/or a dispensing device that can dispense three separate fluids to a common mixing tip or chamber to form the desired single instantly-formed gel. The dispensing device can be formed with three compartments which can be detached containers or integrated chambers in the device that provide triple reservoirs for the three compositions. The apparatus disclosed further can comprise pump or vacuum means to simultaneously withdraw fluid from each compartment, via separate draw pipes, and discharge the fluid to a common mixing tip or chamber. In the case where the pH sensitive rheology modifying polymer is in solid form, a dispensing device can also dispense two separated fluid parts. For example, the first composition may comprise an acidic solution of a pH modifying agent, the second solution may comprise an alkaline solution, and the third composition may comprise a functional ingredient. The third composition may comprise one part of powdered pH sensitive rheology modifier, which is added to a common mixing tip or chamber to form a gel.

According to an embodiment, the three-component kit or system comprises a first composition with an acidic solution of non-hazardous and pH-sensitive rheology modifying polymer(s) like Carbopol, HASE, and/or HEC polymers, with or without low pH (2-4) compatible functional or inert chemical(s), depending on what other function(s) is provided for the product. The three-component kit or system comprises a second composition comprising an alkaline pH modifying agent such as potassium hydroxide, sodium hydroxide, and/or soda, with or without an alkaline solution compatible functional or inert agents, depending on what other function(s) is provided for the product. The three-component kit or system comprises a third composition comprising flavor(s), solvent(s), surfactant(s), and/or other chemical(s), depending on what other function(s) is provided for the product.

Referring to TABLE 2 is one possible embodiment for a three-component kit or system, with a first composition, a second composition, and a third composition. According to an embodiment, the mix ratio of components in TABLE 2 is 1:1:1 (v/v/v) to create a final instantly formed artificial saliva or internal ultrasound gel with ˜45,000 cps @pH 6.5-8.5, 10 rpm and acoustic impedance around 1.5 MRayl, although other variations are possible. The compositions are provided only as an example, and it will be understood that it does not limit the scope of the methods and kits described or otherwise envisioned herein.

TABLE 2
Possible components of a three-component kit or system.

| Ingredient | |
|---|---|
| | % w/w in the |
| | second composition |
| DI Water | 99.31 |
| Potassium Hydroxide | 0.69 |
| | % w/w in the |
| | first composition |
| DI Water | 82.00 |
| Xylitol | 3.00 |
| ACULY 28 | 15.00 |
| | % w/w in the |
| | third composition |
| DI Water | 30.85 |
| Propylene Glycol | 27.00 |
| Glycerin | 42.00 |
| Sodium Lauryl Sulfate (30%) | 0.135 |
| Nat. Peppermint Oil PE-5523 | 0.015 |

According to an embodiment, the single instantly-formed gel is formed by combining four different compositions via a complete mixing through a mixing tip or chamber. Other functional ingredients can be formulated into one part in the third and/or fourth composition. The mixing ratio of each part will be determined by the desired function(s) of the product and the formulation of each ingredient part. The dosing system for the four parts or multi-part composition of the on-site instant artificial saliva gel and/or internal ultrasound gel-forming device can dispense functional ingredients similar to a soda fountain. This system will accurately control dosage of each ingredient. Each ingredient in this approach can be supplied in cartridge form to make it easier for dosing control, replacement, change, or instant multi-flavor or fragrance choice during application.

According to an embodiment, the four-component kit or system comprises a first composition with an acidic solution of non-hazardous and pH-sensitive rheology modifying polymer(s) like Carbopol, HASE, and/or HEC polymers, with or without low pH (2-4) compatible functional or inert chemical(s), depending on what other function(s) is provided for the product. The four-component kit or system comprises a second composition comprising an alkaline pH modifying agent such as potassium hydroxide, sodium hydroxide, and/or soda, with or without an alkaline solution compatible functional or inert agents, depending on what other function(s) is provided for the product. The four-component kit or system comprises a third composition comprising a flavor(s), solvent(s), surfactant(s), and/or other chemical(s), depending on what other function(s) is provided for the product. The four-component kit or system comprises a fourth composition comprising a fourth composition comprising a special functional agent, depending on what other function(s) is provided for the product.

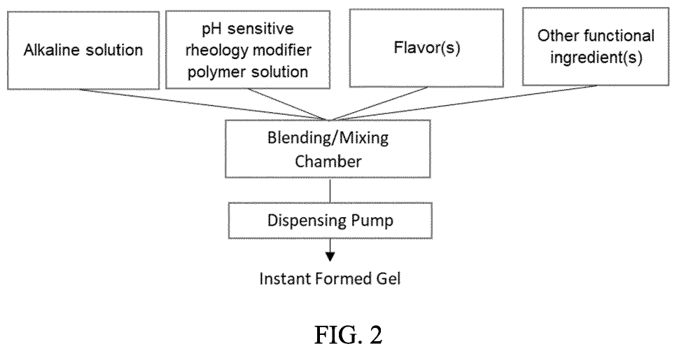

According to an embodiment, the dosing/dispensing system can be designed into a touchscreen vending machine for on-site instantly gel production machine for dental or ultrasound applications. Since flavor or smell plays an important role in consumer product preference, touchscreen options of gel vending machine could be designed as flavor or fragrance choices. For the dispenser users, once the base station is set and common elements are cartridge-like components, the only requirement is shipping and receiving the common components needed for ultrasound gels or artificial saliva gels. FIG. 2 is a schematic representation of one embodiment of such a system.

According to an embodiment, each ingredient used in a final composition of the method or kit is at least compatible with each other at pH 6-8 and temperature ≤approximately 40° C. The final pH of instantly formed artificial saliva gel and ultrasound gel of all ingredients can be within pH 6-8, and preferably pH 6.5-7.5 for human applications.

Referring to TABLE 3 are possible embodiments for a two-, three, and four-component kit or system. The compositions are provided only as an example, and it will be understood that it does not limit the scope of the methods and kits described or otherwise envisioned herein.

TABLE 3
Possible components
Composition of a Two-Component System

| | |
|---|---|
| Second | An alkaline pH modifying agent solution such as potassium hydroxide, |
| component | sodium hydroxide, and/or soda ash in solution, with or without an alkaline |
| | solution compatible functional or inert agents, depending on whether flavor or |
| | other special function is desired |
| First | An acidic solution of non-hazardous & pH sensitive rheology modifying |
| component | polymer(s) like Carbopol, HASE, HEC polymers, and/or other(s), plus low |
| | pH (2-4) compatible flavor(s), solvent(s), surfactant(s), and/or other |
| | functional or inert chemical(s), depending on what other function(s) is desired |

Composition of a Three-Component System

| | |
|---|---|
| Second | An alkaline pH modifying agent solution such as potassium hydroxide, |
| component | sodium hydroxide, and/or soda ash in solution, with or without an alkaline |
| | solution compatible functional or inert agents, depending on whether flavor or |
| | other special function is desired |
| First | An acidic solution of non-hazardous & pH sensitive rheology modifying |
| component | polymer(s) like Carbopol, HASE, HEC polymers, and/or other(s), plus low |
| | pH (2-4) compatible flavor(s), solvent(s), surfactant(s), and/or other |
| | functional or inert chemical(s), depending on what other function(s) is desired |
| Third | A solution of flavor(s), solvent(s), surfactant(s), and/or other chemical(s), |
| component | depending on what other function(s) is provided for the product. |

Composition of a Four-Component System

| | |
|---|---|
| Second | An alkaline pH modifying agent solution such as potassium hydroxide, |
| component | sodium hydroxide, and/or soda ash in solution, with or without an alkaline |
| | solution compatible functional or inert agents, depending on whether flavor or |
| | other special function is desired |
| First | An acidic solution of non-hazardous & pH sensitive rheology modifying |
| component | polymer(s) like Carbopol, HASE, HEC polymers, and/or other(s), plus low |
| | pH (2-4) compatible flavor(s), solvent(s), surfactant(s), and/or other |
| | functional or inert chemical(s), depending on what other function(s) is desired |
| Third | A solution of flavor(s), solvent(s), surfactant(s), and/or other chemical(s), |
| component | depending on what function(s) is desired |
| Fourth | Solution of special functional agent, depending on what other function(s) is |
| component | desired |

Composition of a Multi-Component System

| | |
|---|---|
| Second | An alkaline pH modifying agent solution such as potassium hydroxide, |
| component | sodium hydroxide, and/or soda ash in solution, with or without an alkaline |
| | solution compatible functional or inert agents, depending on whether flavor or |
| | other special function is desired |
| First | An acidic solution of non-hazardous & pH sensitive rheology modifying |
| component | polymer(s) like Carbopol, HASE, HEC polymers, and/or other(s), plus low |
| | pH (2-4) compatible flavor(s), solvent(s), surfactant(s), and/or other |
| | functional or inert chemical(s), depending on what other function(s) is desired |
| Third | Solution of flavor(s) |
| component | |
| Fourth | Solution of special functional agent(s) |
| component | |
| Fifth | Solution of solvent, surfactant(s), and/or other(s) |
| component | |
| Nth | Solution of ingredient N of the formula. |
| component | |

According to an embodiment, depending on the number of parts in composition, the dosing system/device varies accordingly.

According to an embodiment, the components are provided and stored as two separate parts, allowing for provision of the two parts as single-phase compositions maintained at different pH levels predetermined based on the strength (%) of the components present in a respective part. This enables the kit to achieve long storage stability or shelf life with no preservative or stabilizer added. According to an embodiment, lack of preservative or stabilizer is possible due to the lack of common essential nutrients required for the growth of microorganisms, such as carbon, hydrogen, nitrogen, oxygen, phosphorus, and sulfur. Both the first composition and the second composition lack nitrogen, phosphorus, and sulfur, which are important essential nutrients in making proteins, nucleic acids, and cellular energy transfers. Without sufficient phosphate, for example, microorganisms will not grow.

According to an embodiment, lack of preservative or stabilizer is also possible due to the lack of essential trace elements such as iron, copper, manganese, nickel, and zinc. These elements, which are essential for the synthesis of enzymes used for microorganism metabolism, are mostly absent from the formula.

According to an embodiment, lack of preservative or stabilizer is further possible due to the extremely low and unfavorable pH, such as pH<4.0, of the first composition, and the extremely high and unfavorable pH, such as pH>11.0, of the second composition. The effect of pH on microorganism growth is very important. Most bacteria prefer neutral pH, such as 6.5-7.5. Molds and yeast grow in wider pH range, but prefer pH between 5 and 6.

According to an embodiment, lack of preservative or stabilizer is also possible due to anti-microbial ingredients in the kit. For example, solvents such as propylene glycol and glycerin have anti-microbial properties, as do essential oils of flavoring agents like eugenol (antiseptic) and natural peppermint oil (antibacterial). Xylitol is non-fermentable sugar alcohol flavoring agent and has anti-microbial property.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for generating an instantly-forming artificial saliva gel or an ultrasound gel prior to use of the gel, comprising:
providing a first composition comprising a pH-sensitive polymer, wherein the first composition is maintained at an acidic pH;
providing a second composition comprising a pH agent configured to maintain the second composition at an alkaline pH; and
combining the first composition and the second composition to generate a single instantly-formed gel, wherein the pH of the single instantly-formed gel is at a level required to increase viscosity of the pH-sensitive polymer to a level suitable for an artificial saliva gel or an ultrasound gel;
wherein either the first composition or second composition comprises a solvent, and wherein the acidic pH of the first composition and the alkaline pH of the second composition are configured such that the pH of the single instantly-formed gel is between approximately 6.5 and 7.5.

2. The method of claim 1, further comprising:
providing a third composition comprising a flavor agent and/or a surfactant; and
wherein said combining step further comprises combining the third composition with the first composition and the second composition.

3. The method of claim 1, further comprising:
providing a third composition, wherein the third composition rather than the first composition or the second composition comprises the solvent; and
wherein said combining step further comprises combining the third composition with the first composition and the second composition.

4. The method of claim 1, wherein the pH agent is a hydroxide, carbonate, bicarbonate, silicate, phosphate, and/or variations thereof.

5. The method of claim 1, wherein either the first composition or second composition comprises a flavor agent, a surfactant, a sweetening agent, and/or a fragrance.

6. The method of claim 5, wherein the flavor agent is xylitol, menthol, eugenol, and/or an essential oil.

7. The method of claim 5, wherein the surfactant is SLS.

8. The method of claim 1, wherein the solvent is propylene glycol and/or glycerin.

9. The method of claim 1, wherein the first composition and the second composition do not comprise a preservative.

10. A kit configured to generate an instantly-formed artificial saliva gel or an ultrasound gel just prior to use of the gel, comprising:
a first composition comprising a pH-sensitive polymer, wherein the first composition is maintained at an acidic pH; and
a second composition comprising a pH agent configured to maintain the second composition at an alkaline pH;
wherein combining the first composition and the second composition via a mixing tip or chamber generates a single instantly-formed gel, wherein the pH of the single solution is at a level required to increase viscosity of the pH-sensitive polymer to a level suitable for an artificial saliva gel or an ultrasound gel;
wherein either the first composition or second composition comprises a solvent, and wherein the acidic pH of the first composition and the alkaline pH of the second composition are configured such that the pH of the single instantly-formed gel is between approximately 6.5 and 7.5.

11. The kit of claim 10, further comprising:
a third composition comprising a flavor agent and/or a surfactant; and
wherein combining the first composition, the second composition, and the third composition via the mixing tip or chamber generates the single instantly-formed gel.

12. The kit of claim 10, wherein the pH agent is a hydroxide, carbonate, bicarbonate, silicate, phosphate, and/or variations thereof.

13. The kit of claim 10, wherein either the first composition or second composition comprises a flavor agent, a surfactant, a sweetening agent, and/or a fragrance.

14. The kit of claim 13, wherein the flavor agent is xylitol, menthol, eugenol, and/or an essential oil.

15. The kit of claim 10, wherein the solvent is propylene glycol and/or glycerin.

16. The kit of claim 10, wherein the first composition and the second composition do not comprise a preservative.
